(19) United States
(12) Patent Application Publication
(10) Pub. No.: US 2017/0079517 A1
(43) Pub. Date: Mar. 23, 2017

(54) INSTRUMENT FOR EARLY DIAGNOSIS OF LARYNX CANCER

(71) Applicant: MITOS MEDIKAL TEKNOLOJILER SANAYI VE TICARET ANONIM SIRKETI, Istanbul (TR)

(72) Inventors: Murat ÖZCAN, Istanbul (TR); Mehmet ÇAYÖREN, Istanbul (TR); Ismail KURTOGLU, Istanbul (TR); Mustafa Tunaya KALKAN, Istanbul (TR); Ibrahim AKDUMAN, Istanbul (TR); Güray Ali CANLI, Istanbul (TR)

(21) Appl. No.: 15/307,962

(22) PCT Filed: May 4, 2015
(86) PCT No.: PCT/TR2015/000187

§ 371 Date: Oct. 31, 2016

(30) Foreign Application Priority Data
May 2, 2014 (TR) 2014/04917

(51) Int. Cl. A61B 1/247; A61B 1/267; A61B 1/06; A61B 1/00; A61B 1/05

(52) U.S. Cl. CPC A61B 1/247; A61B 1/00016; A61B 1/00034; A61B 1/05; A61B 1/0676; A61B 1/0684; A61B 1/00018; A61B 1/00105; A61B 1/2673

(57) ABSTRACT

This invention is regarding an instrument (1) that is intended for use in the medical field for imaging and early diagnosis of larynx cancer. The instrument described in the invention involves LED illumination (1.1.3), which are driven at a frequency that is related to the fundamental frequency of the patient sound, a microphone (1.1.4) to acquire the fundamental frequency of the patient; an indirect laryngoscope section (1.1) that consists of a semi transparent mirror (1.1.1), and a power section (1.3) that provides the electrical current that is necessary to power up the instrument for early diagnosis of larynx cancer.

### FIELD OF THE INVENTION

This invention is concerned with developing an instrument for early diagnosis of larynx cancer, which is to be used in the field of medicine.

### BACKGROUND OF THE INVENTION

Larynx cancer constitutes 3% among all cancer types. The larynx is located underneath the throat; that is, before the esophagus and above the lungs. Its primary function is to prevent food from going into the lungs, aiding speech and breathing. The larynx is formed of three parts, the glottis where vocal cords are located, the supraglottis that is above the vocal cords and the subglottis that is below the vocal cords. Larynx cancer predominantly occurs (60%) on the vocal cords.

The symptoms of this disease varies based on its location at the throat. If the cancer occurs on the vocal cords, the first symptom arises as a voice problem and as a hoarseness of the voice. The most major symptom is voice hoarseness that does not heal. Often, the larynx cancer is mixed for pharyngitis, laryngitis and cold, by the doctor.

Likewise other cancer types, early diagnosis is the most crucial factor in the larynx cancer. When diagnosed early, larynx cancer is likely to be treated. However, the entire larynx has to be removed when diagnosed late. Since early diagnosis is crucial, the symptoms should not be underestimated. Thus, doctors should examine, considering the possibility for larynx cancer.

Today, multiple methods exist for early diagnostics. Mirrored (indirect laryngoscope) or endoscopic (direct) examination of the larynx are the major examination methods. Furthermore, swelling of the neck is checked during examination. For accurate diagnosis, suspected tissue (biopsy) is dissected for pathological investigation.

In the larynx cancer, radiological investigations are performed to determine the degree and spreading of the disease. Computerized Tomography (CT), Magnetic Resonance Imaging (MRI), and ultrasound imaging are commonly used for this purpose. Further investigations are performed to check metastasis.

Another method that is being employed today is the stroboscopic laryngoscope. Stroboscopic examination has recently become popular in the clinic. Stroboscopy is a crucial technique to monitor rapid movement of the vibratory patterns of the vocal cords, which is not possible to observe with the human eye. According to Talbot's law, the retina can sense up to 5 images/second. Therefore, images that are imaged at the Retina for less than 0.2 seconds (5 images/second) are observed to be continuous and combined at the ocular cortex to form a continuous motion. During stroboscopic imaging, larynx is commonly imaged using a xenon light. The characteristic of xenon light is to allow rapid pulsed illumination. In this context, larynx can be observed only at 1/1000 second intervals. The short images of the larynx, sampled at multiple points of various vibratory cycles, come together to make observation of vocal cord tissue motion possible.

In a modern stroboscopic setup, laryngeal vibrations are observed with a microphone that is wrapped around the neck that is used to control the pulsing of the xenon light. When the sampling rate of the laryngeal image becomes out-of-phase with the vibrating frequency of the vocal cords, the laryngeal tissue is observed to be moving. If sampling rate happens to be at the same phase with the vibrating frequency of the vocal cords, the laryngeal tissue is observed to be stationary.

Stroboscopy allows observing the vibrating movement of the vocal cords, which is not possible with a continuous light source. As described before, vibratory activity is necessary for producing sound. Accordingly, the observer may monitor how small lesions effect the vibratory pattern using a stroboscope, from which the significance of the lesion could be judged.

However, stroboscopic laryngoscopes are costly and can only be operated by experts. The stroboscopic laryngoscopes have a connection cable, connected to the data acquisition unit, and use external light sources. These light sources are usually tied to the forehead of the doctor or mounted on a table. On the other hand, the microphone is wrapped around patients neck. All components are connected to each other through cables, where the fundamental frequency of the patient is displayed at the monitor unit. With a foot-pedal, the physician can adjust the fundamental frequency measured and observed of the larynx at the preferred light source.

Despite being high cost, this system is also bulky and not practical. A practitioner can not perform a stroboscopic laryngoscope examination to every patient that refers due to voice hoarseness. At instants where the practitioner suspects a lesion, the patient is transferred to an otorhinolaryngology specialist. This may even end up in patient death, as early diagnosis is crucial for patients with the larynx cancer.

One other disadvantage of stroboscopic laryngoscopes is that they are only available at the office of otorhinolaryngology specialists, however, doctors from internal medicine and odontology background can not access this instrument and easily perform the examination. This hampers the ability of early diagnosis of larynx cancers as well.

Another disadvantage of the system is its complexity, as the system contains multiple components including a light source, a microphone, a foot-pedal, a monitor and cables. Setting up of the system is time costly and requires control of its numerous elements.

Another disadvantage of the system is that it is bulky due to having multiple components. Thus, the system can not fit in every doctor's office and typically is harbored in research hospital. This in turn, becomes a bottleneck in the availability of the instrument, and thus the early diagnosis of larynx cancer.

In the literature, the patent application no: CN202950636 envisions a system having indirect laryngoscope that has a handle with a steam impermeable mirror on top. The light source of the indirect laryngoscope is placed on the front side of the handle, close to the position of the mirror which is tolerant to fog. When compared with the previous technique, the light source is placed on the handle. In accordance with this application, the doctor can operate the laryngoscope using one hand, and hold the patients head and lower jaw with the other for assistance.

Another relevant patent application (CN201542603) envisions an indirect laryngoscope consisting of a lens and a handle. 4 light sources are embedded around the lens. The handle is made out of lead, where the frontal end of the handle consists of a mobile edge, a battery and a trigger. The mobile edge is designed to tilt between 90-180 degrees, which is appropriate to monitor the patients larynx.

The above inventions aim to overcome certain disadvantages. However, the light sources are designed only to illuminate the larynx, and lack stroboscopic capability, hampering interpretation of the symptoms on the vocal cords. Furthermore, the mentioned inventions do not involve a microphone, therefore requires an external microphone to be placed around the neck.

In conclusion, advances are being made towards early diagnosis of larynx cancer. Further novel instrumental development is required to mitigate the disadvantages that are mentioned above of current systems.

### OBJECTIVE OF THE INVENTION

This invention is concerned with a novel instrument , which aims to meet the above mentioned requirements while overcoming all disadvantages, and bringing further advantages for early diagnosis of larynx cancer.

One aim of the invention is to assemble all the components, mentioned above, onto a single body. Stroboscopy could be formed through embedding multiple LED light sources, a microphone, a knob, and a microcamera into a wireless hand-mirror, eliminating the necessity of using wires for data transfer. This allows the doctor to easily control this mechanism, which is gathered on a single body, using a single hand only. In this sense, the proposed instrument will be more handy and practical for early diagnosis of cancer.

One other aim of the invention is to bring the cost of this device for early diagnosis of larynx cancer down, having all entities embedded in a single body. With lowered cost, not only otorhinolaryngologist offices will harbor these devices, but it can further be made available at any doctor's office. Mouth and larynx examinations are not only performed by otorhinolaryngologists. Dentists, Internal medicine specialists and family practitioners also perform these examinations, by using a larynx mirror. Owing to its low cost and practical usage, doctors will be able to use the developed stroboscopic laryngoscope instead of a larynx mirror, in their examinations.

As dentists, internal medicine specialists and family practitioners are not specialized on larynx, they may not be able to interpret lesion in this area. One other aim of the proposed instrument is to store the images of the examined regions (plikavocalis base, etc.) and transfer to the external monitor using wireless RF technology. The images that are transferred to the external monitor can be gathered in a portal, allowing a doctor who is not specialized in laryngology to share the images with laryngologists through this portal. In turn this will enable the laryngologist to diagnose the larynx cancer correctly based on its early stage symptoms very similar to those of cold, upper respiratory and tonsillar infections.

Another goal if the invention is the capability to store glottis images in video format, where these stored videos could be compared with the videos of prior examinations of the same patient for consultation. Stored images will provide changes of the glottal vibratory pattern over days, weeks and years. The comparison of the vibratory pattern over time will reveal the effects of surgical and medical interventions on the tissue.

Another goal of the invention is to enable practical and quick examination of the patient, without feeling tired. Vocal cords will be examined similar to a dental examination using the proposed wireless instrument, through requesting the patient to conduct ‘A’ and ‘Hi’ sounds.

Structural and characteristic features, along with all the advantages of the device will be detailed in the below descriptions and figures for clarity.

### BRIEF DESCRIPTION OF FIGURES

Below figures and captions should be considered to reveal the full advantages, structuring, and add-on's of the current invention in its assessment:

FIG. 1; Perspective view of the instrument that is capable of early diagnosis of larynx cancer.

FIG. 2; Inside view of the instrument that is capable of early diagnosis of larynx cancer, showing the internal cables.

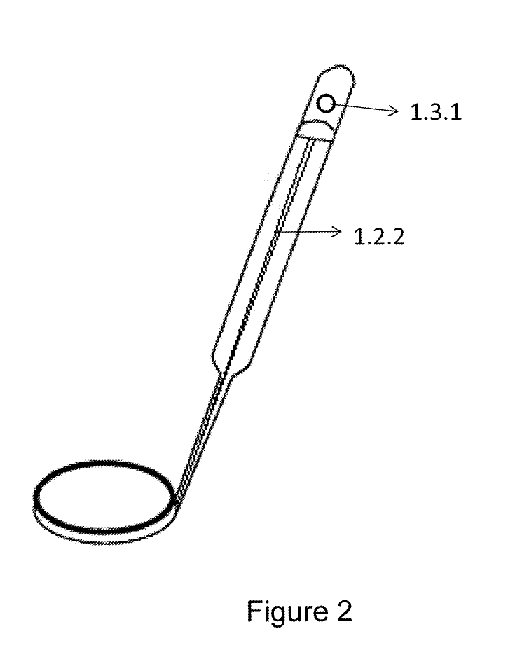

FIG. 3; Detailed view of the indirect laryngoscope section.

FIG. 4; View of modular separation of the indirect laryngoscope, handle and power sections of the instrument that is capable of early diagnosis of larynx cancer.

FIG. 5; View of the charging (adaptor) input of the instrument that is capable of early diagnosis of larynx cancer.

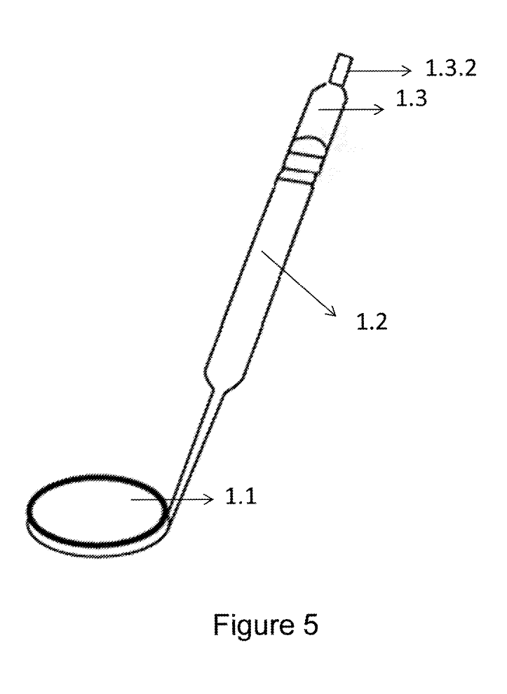

FIG. 6; View of the power cable, that is located at the power section of the instrument that is capable of early diagnosis of larynx cancer.

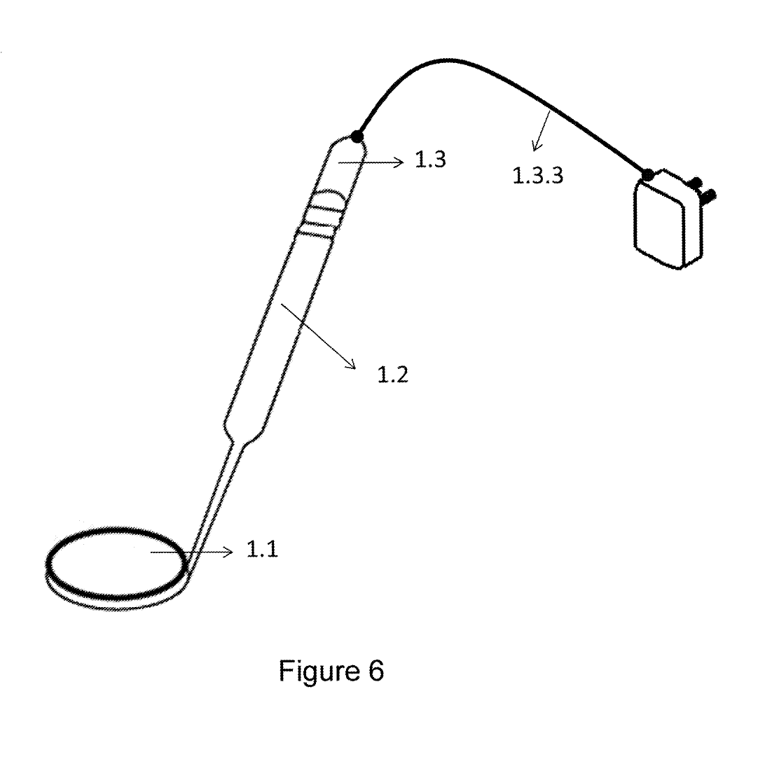

### REFERENCE NUMBERS

1. Instrument that is capable of early diagnosis of larynx cancer
   1.1. Indirect laryngoscope section
      1.1.1. Semi-transparent mirror
      1.1.2. Camera
      1.1.3. LED's
      1.1.4. Microphone
   1.2. Handle section
      1.2.1. Frequency adjustment knob
      1.2.2. Electronic cable
      1.2.3. Circuit
   1.3. Power section
      1.3.1. RF transmitter
      1.3.2. Charge input
      1.3.3. Power cable
      1.3.4. Chargeable batteries

### DETAILED DESCRIPTION OF THE INVENTION

In this detailed description, desired modes/structuring of the instrument for early diagnosis of larynx cancer (1) are given for clarity without any limitations.

This invention is concerned with an indirect laryngoscope instrument (1) for early diagnosis of larynx cancer, to be used in the field of medicine for imaging the larynx.

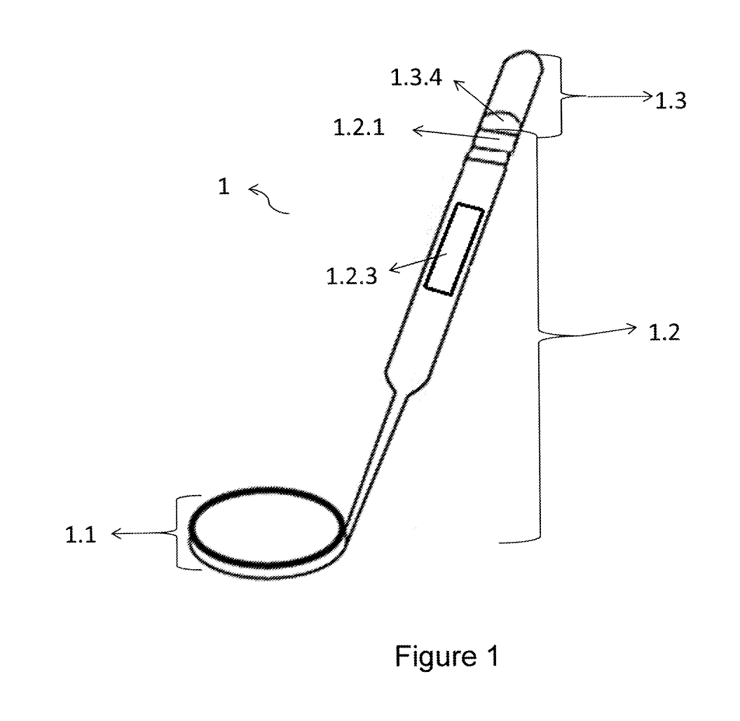

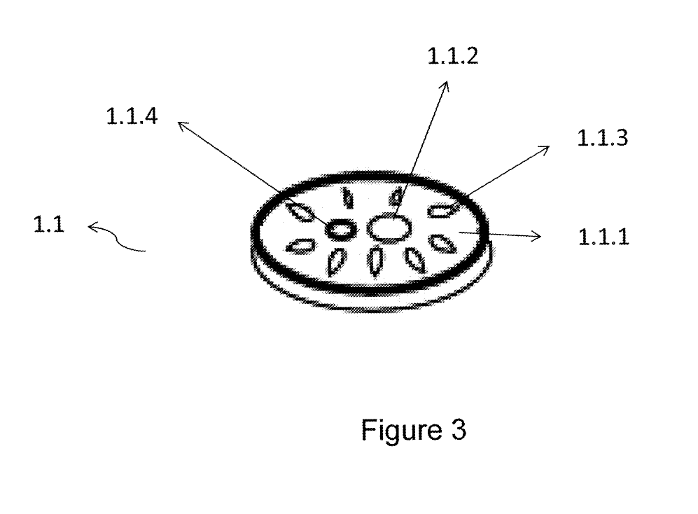

FIG. 1 depicts a three-dimensional view of the instrument for early diagnosis of larynx cancer (1). This invention, as seen in FIG. 3 in its indirect laryngoscope section (1.1), involves a camera (1.1.2) at the center of the semi-transparent mirror (1.1.1). During the examination, the doctor is able to image the larynx tissue using the camera (1.1.2) and transmit using the RF transmitter (1.3.1) to the external monitor. The larynx images of the patients that are acquired with the camera (1.1.2), can be gathered in a portal. Thus, the images that are collected at the portal are shared with specialists for correct diagnosis of the symptoms. For example, tissue images that are gathered during a dental examination are also transferred to an otorhinolaryngologist, thus enabling early diagnosis and treatment of the possible larynx cancer, preventing metasthasis.

The invention includes LED lights (1.1.3) at the periphery of the semi-transparent mirror (1.1.1) in the indirect laryngoscope section (1.1). LED's (1.1.3) are stroboscopic light sources. Main advantages of the instrument for early diagnosis of larynx cancer (1) is the ability of the long-lasting LED lights to act as a continuous as well as stroboscopic light source, smart and low decibel, having a pure white illumination at 6300° K. The instrument for early diagnosis of larynx cancer (1) is locked to the fundamental frequency of the sound that is detected with the microphone (1.1.4) and calculated with the known techniques. The instrument for early diagnosis of larynx cancer (1) deduces the fundamental frequency as follows: A circuit (1.2.3) is located in the handle section (1.2). This circuit (1.2.3) could either be an electronic circuit or a PLL (Phase-locked-loop). The circuit (1.2.3) measures the fundamental frequency through the software embedded inside the microcontroller or using PLL. LED lights (1.1.3) are illuminated with the measured frequency. Owing to the stroboscopic nature of the LED's (1.1.3) the doctor observes the vocal cords as stationary during larynx examination.

The invention comprises a microphone (1.1.4) at the indirect laryngoscope section (1.1). In an alternative arrangement, the microphone (1.1.4) is located in the handle section (1.2). The physician carefully inserts the instrument for early diagnosis of larynx cancer (1) into patient's mouth. The larynx is first observed while the patient is at rest. Then, the vocal cords are examined while the patient is asked to make ‘A’ and ‘ Hi’ sounds, during which the microphone (1.1.4) automatically deduces the fundamental frequency of the sounds and the operation frequency of the LED (1.1.3) is adjusted accordingly. The number of cycles that the glottis (inter-vocal cord tissue) makes is denoted as the fundamental (basal) frequency. The fundamental frequency during speech is typically in the 100-150 Hz for men and 150-250 Hz for women. The instrument for early diagnosis of larynx cancer (1) first determines the fundamental frequency of sound with techniques known by those skilled in the art. Then, the instrument adjusts illumination frequency of LEDs (1.1.3) according to the determined frequency again with techniques known by those skilled in the art.

The invention includes a frequency adjustment knob (1.2.1) at the handle section (1.2). The frequency adjustment knob (1.2.1), is used for tuning the LED (1.1.3) frequency in accordance with the fundamental frequency of the voice that is captured through the microphone (1.1.4). With the frequency adjustment knob (1.2.1), the fundamental frequency can be adjusted as −10, . . . −2, −1, 0, +1 or +2 . . . , +10. For example, if the frequency adjustment knob (1.2.1) is set to 0, the LED frequencies are tuned to the fundamental frequency of the sound, captured through the microphone (1.1.4). On the other hand, if the frequency adjustment knob (1.2.1) is adjusted to +1, the instrument for early diagnosis of larynx cancer (1) adjusts its frequency to 1 Hz above that of the fundamental frequency that is captured via the microphone (1.1.4). When the frequency adjustment knob (1.2.1) is set to 0, the physician observes the vocal cords as stationary. When adjusted to −1, −2, +1 or +2, the doctor observes the vocal cords to be moving very slowly. The doctor is free to set the frequency adjustment knob (1.2.1) to a fundamental frequency with which s/he is comfortable to work with to examine vocal cord movement. For example, if the patients fundamental frequency is observed to be 300 Hz, when asked to make “A” or “Hi” sound, and if the frequency adjustment knob (1.2.1) is set to 0, the device for early diagnosis of larynx cancer (1) automatically sets its frequency to 300 Hz. But, if the frequency adjustment knob (1.2.1) is set to +1, the instrument for early diagnosis of larynx cancer automatically sets its frequency to 301 Hz. When the measured frequency of the patient is 300 Hz and the frequency adjustment knob (1.2.1) is set to 0, the physician observes the vocal cords as stationary. If the measured frequency of the patient is 300 Hz and the frequency adjustment knob (1.2.1) is set to +1, the instrument for early diagnosis of larynx cancer (1) operates at 301 Hz and the doctor observes the vocal cords as moving.

The invention involves an RF transmitter (1.3.1) in its power section (1.3). With the RF transmitter (1.3.1) the images acquired with the camera (1.1.2) can be transferred to an external monitor. The larynx images that are acquired with the camera (1.1.2) can be gathered in a portal. This way, as mentioned earlier, the images that are gathered in the portal can be shared with larynx experts for precise diagnosis.

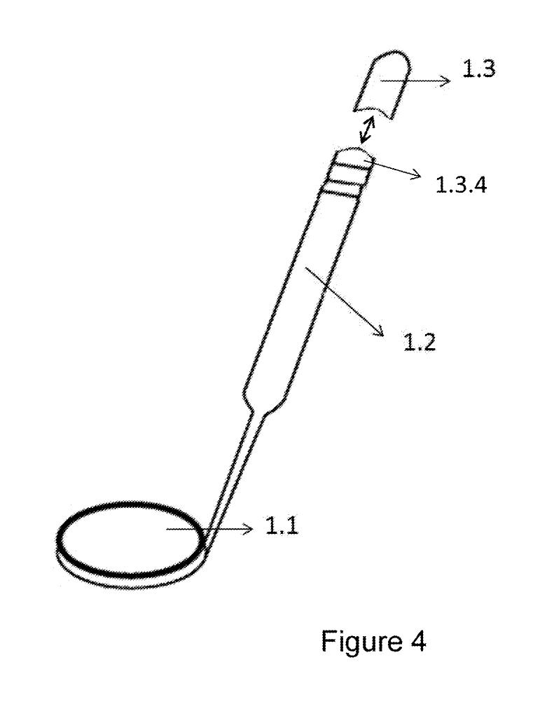

The instrument for early diagnosis of larynx cancer (1), consists of an indirect laryngoscope section (1.1), the handle (1.2) and the power (1.3) sections. These units can be integrated, or alternatively combined in a modular fashion as shown in FIG. 4. That is, while the indirect laryngoscope (1.1) and the handle (1.2 sections) are integrated, the power section (1.3) is modular and can be separated. The benefit is to be able to isolate the power section (1.3) from the indirect laryngoscope (1.1) and handle (1.3) for charging also to be able to sterilize the indirect laryngoscope (1.1) and the handle (1.2) sections, without the power (1.3) section. The physician's office can keep a single power unit (1.3), while having 10 or more of the indirect laryngoscope (1.1) and handle (1.2) units. After the examination, the physician separates the integrated indirect laryngoscope (1.1) and handle (1.2) section from the power section (1.3) for sterilization, while connecting a sterilized pair of integrated indirect laryngoscope (1.1) and handle (1.2) sections to the power unit (1.3) that he has just used, for the next patient.

In another aspect, device modularity embodiment involves, rechargeable batteries (1.3.4) that are located in between the connection point of the power section (1.3) and the handle section (1.2). After being separated from the entire mechanism, the rechargeable batteries (1.3.4) are charged and re-inserted between the power (1.3) and handle (1.2) sections, for the continuity of electronic operation. In another embodiment, the instrument for early diagnosis of larynx cancer (1) is composed of an integrated indirect laryngoscope (1.1), the handle (1.2) and the power sections (1.3). in this alternative form, the instrument for early diagnosis of larynx cancer (1) is sterilized as a whole. The power input (1.3.2) that is dedicated for charging the device (1.3.2) stays outside the sterilization boundary to prevent oxidation. The power input (1.3.2) is located at the top of the power unit (1.3)

The interconnection between the power section (1.3) and the LEDs (1.1.3), the camera (1.1.2), the microphone (1.1.4) and the frequency adjustment knob (1.2.1) located in the indirect laryngoscope section (1.1) are maintained via electronic cables (1.2.2).

The instrument for early diagnosis of larynx cancer (1) can be powered up through the power section (1.3), by rechargeable batteries (1.3.4), through being charged using the power input (1.3.2) or also via a power cable (1.3.3) that can be externally connected.

The application of the instrument for early diagnosis of larynx cancer (1) is as follows:

The physician observes the larynx of the patient by inserting the instrument for the early diagnosis of larynx cancer (1) inside the patient's mouth, through the illuminating the area with the LED lights (1.1.1). Afterwards, the vocal cords are examined through asking the patient to make “A” and “Hi” sound. The microphone (1.1.4) acquires the sound and deduces the fundamental frequency, with which the illumination frequency of the LEDs (1.1.3) of the instrument for early diagnosis of larynx cancer (1) is adjusted using known techniques (Via electronic circuitry or PLL). The physician able to adjust the LED illumination frequency adjustment knob of the instrument for early diagnosis of larynx cancer (1) to one of −10, . . . −2, −1, 0, +1 or +2 . . . , +10 settings. The physician is able to understand and diagnose the larynx through either observing the vocal cords at matched LED illumination frequency and voice fundamental frequency, or at slightly unmatched frequencies to observe the vibration of the vocal cords in slow motion.

1. An instrument (1) having semi-transparent mirror (1.1.1) for early diagnosis of larynx cancer, characterized in comprising
indirect laryngoscope section (1.1) which comprises stroboscopic LED lights (1.1.3), that are driven at a frequency through the fundamental frequency of the patient sound,
a microphone (1.1.4) to acquire the fundamental frequency of the patient when the patient makes a sound,
handle section (1,2) through which electronic cables (1.2.2) pass,
power section (1.3) providing the electrical current to LED's (1.1.3), camera (1.1.2) and microphone (1.1.4).

2. The instrument (1) for early diagnosis of larynx cancer according to claim 1, characterized in that said indirect laryngoscope section (1.1) comprises
a camera (1.1.2) which allows the doctor to simultaneously image the examined data while examining the larynx of the patient.

3. The instrument (1) for early diagnosis of larynx cancer according to claim 1, characterized in comprising
a power cable (1.3.3) connection that provides transfer of the external electrical current to the instrument for early diagnosis of larynx cancer (1).

4. The instrument (1) for early diagnosis of larynx cancer according to claim 1, characterized in comprising at the handle section (1.2)
frequency adjustment knob (1.2.1) which provides adjusting the instrument (1) for early diagnosis of larynx cancer, that is tuned to the fundamental frequency of the patient, as −10, . . . , −2, −1, 0, +1 or +2, . . . , +10, in order to bring the same into the required fundamental frequency.

5. The instrument (1) for early diagnosis of larynx cancer according to claim 1, characterized in comprising in said power section (1.3)
RF transmitter (1.3.1) that transfers the images acquired with the camera (1.1.2) to an external monitor.

6. The instrument (1) for early diagnosis of larynx cancer according to claims 1 to 4, characterized in that the images that are transferred to an external monitor by the RF transmitter (1.3.1) are gathered in a portal.

7. The instrument (1) for early diagnosis of larynx cancer according to claim 1, characterized in comprising indirect laryngoscope section (1.1), handle section (1.2) and power section (1.3) in an integrated fashion such that those are not separated from each other.

8. The instrument (1) for early diagnosis of larynx cancer according to claim 1, characterized in comprising power section (1.3) which is separated modularly from the indirect laryngoscope section (1.1) and the handle section (1.2) that are integrated, and which provides charging of the instrument (1) for early diagnosis of larynx cancer.

9. The instrument (1) for early diagnosis of larynx cancer according to claim 1, characterized in comprising chargeable battery (1.3.4) that is located between the integrated indirect laryngoscope section (1.1) and the handle section (1.2) and the modularly separated power section (1.3).

10. The instrument (1) for early diagnosis of larynx cancer according to claim 1, characterized in comprising charge input (1.3.2) located at the top of the wireless power section (1.3) which provides the instrument (1) for early diagnosis of larynx cancer to charge.

11. The instrument (1) for early diagnosis of larynx cancer according to claim 1, characterized in that microphone (1.1.4) can be located in the indirect laryngoscope section (1.1) or in the handle section (1.2).

12. The instrument (1) for early diagnosis of larynx cancer according to claim 1, characterized in comprising electronic cables (1.2.2) which are located in the handle section (1.2) and which provide electronic connection between the power section (1.3) and the LED lights (1.1.3), camera (1.1.2), microphone (1.1.4) and frequency adjustment knob (1.2.1) in the indirect laryngoscope section (1.1).

13. A method for early diagnosis of larynx cancer, characterized in comprising the process steps of
observing the larynx of the patient through the semi-transparent mirror (1.1.1) by means of the LED's (1.1.3) located in the indirect laryngoscope section (1.1) of the instrument (1) for early diagnosis of larynx cancer,
measuring the fundamental frequency of the patient by acquiring the sound by the microphone (1.1.4) when the patient makes a sound,
adjusting the fundamental frequency of the instrument (1) for early diagnosis of larynx cancer by means of the circuit (1.2.3) located in the handle section (1.2),
lighting LED's (1.1.3) with the same measured fundamental frequency of the instrument (1) for early diagnosis of larynx cancer.

14. The method for early diagnosis of larynx cancer according to claim 13, characterized in comprising the process steps of
acquiring images with the camera (1.1.2) during the observing of the larynx of the patient through the semi-transparent mirror (1.1.1) by means of illuminating the LED's (1.1.3),
transferring the images acquired with the camera (1.1.2) to an external monitor.

15. The method for early diagnosis of larynx cancer according to claim 13, characterized in comprising the process steps of
adjusting the instrument (1) for early diagnosis of larynx cancer to the intended fundamental frequency by means of the frequency adjustment knob (1.2.2) by changing the fundamental frequency of the instrument (1) for early diagnosis of larynx cancer as −1, . . . , 0, +1, 0, +1 or +2, . . . , +10 after adjusting fundamental frequency of the same.
